(19) United States
(12) Patent Application Publication
(10) Pub. No.: US 2016/0228116 A1
(43) Pub. Date: Aug. 11, 2016

(54) INSTRUMENT INTRODUCER

(71) Applicant: Covidien LP, Mansfield, MA (US)

(72) Inventor: Keith Milliman, Port Richey, FL (US)

(21) Appl. No.: 15/132,710

(22) Filed: Apr. 19, 2016

Related U.S. Application Data
(63) Continuation of application No. 11/220,263, filed on Sep. 6, 2005.

(51) Int. Cl. A61B 17/02

(52) U.S. Cl. CPC A61B 17/0218; A61B 17/0293; A61B 2017/0225; A61B 17/068

(57) ABSTRACT

Instrument introducers and methods of using the same are provided to facilitate the introduction of a surgical instrument into a cavity or a body opening of a patient. The instrument introducers include a body portion defining a lumen therethrough, a flexible distal end portion having a distal orifice, a proximal end portion having a proximal orifice, and at least one fold formed in at least the distal end portion.

### CROSS-REFERENCE TO RELATED APPLICATIONS

The present application is a Continuation Application which claims the benefit of and priority to U.S. patent application Ser. No. 11/220,263, filed on Sep. 6, 2005, the entire disclosure of which is hereby incorporated by reference herein.

### BACKGROUND

1. Technical Field

The present disclosure relates to instrument introducers or protective sleeves and methods of using the same and, more particularly, to novel protective sheaths configured to facilitate the introduction of surgical instruments into a cavity, an orifice or a body opening of a patient.

2. Background of Related Art

Surgical instruments configured for remote use inside the body of a patient typically define a central longitudinal axis including a distal end portion and a proximal end portion. The distal end portion of the surgical instrument can include configurations which have either blunt or rounded faces and/or in certain instances include sharper subassemblies including electrosurgical and/or mechanical blades for cutting as well as fasteners for securing tissue portions. As a result of the mechanical complexity of these surgical instruments, the distal end portions of these instruments have been provided with a shell or cover that may partially or fully enclose the distal end portion of the surgical instrument.

For example, presently, various intra-anal surgical instruments, such as intraluminal anastomotic surgical staplers, require insertion into the colon or intestine through the anus. In certain embodiments, these surgical staplers have staple anvil portions removably mounted to a distal end thereof. Generally, the surgical stapler is inserted with the anvil portion attached, however, certain surgical procedures require that the surgical stapler be inserted into the colon or intestine through the anus with the anvil portion removed therefrom.

Typically, in instances where the surgical stapler is to be inserted into the colon or intestine of the patient with the anvil portion mounted to a distal end thereof, the anvil portion is tapered toward the tip and formed to have an atraumatic end, thus facilitating the insertion of the distal end of the surgical instrument. However, in such instances or in instances where the surgical stapler is to be inserted into the colon or intestine without the anvil portion mounted to a distal end thereof, it is desirable to have an instrument and/or accessory which reduces potential irritation and/or trauma to the surrounding tissue which may result from, for example, the substantially squared or non-tapered distal end of the surgical instrument.

Thus, a need exists for a instrument introducer in the form of a sheath, which instrument introducer facilitates the passage of the surgical instrument into the body of the patient and that can be either adapted to be removably mounted on a distal end of the surgical instrument or be used as a separate device adapted to be positioned at least partially into the body of the patient (i.e., into the anus) and wherein the surgical instrument is subsequently positioned into the instrument introducer to facilitate insertion of the surgical instrument into the body of the patient.

### SUMMARY

The present disclosure is directed to instrument introducers configured to facilitate the introduction of surgical instrument into a body opening of a patient. In one aspect of the present disclosure, the instrument introducer includes a body portion having a distal end portion including a distal orifice, and a proximal end portion including a proximal orifice. The body portion defines a lumen for passage of a portion of a surgical instrument therethrough. The distal end portion includes at least one fold formed therein. The distal end portion can have a plurality of folds extending radially thereabout. The plurality of folds can be configured and dimensioned to define a distal end portion having a tapered configuration, for example, a generally conical or frusto-conical shape.

At least the distal end portion of the instrument introducer can be fabricated from at least one or a combination of suitable materials, for example, a polypropylene, a moldable plastic, a thermoformable plastic, a polymer, a urethane, a silicone, a natural rubber, a synthetic rubber, an elastomer, an elastomeric material, and a latex material. The instrument introducer can be fabricated from a material having a durometer reading of about 5A to about 90A, preferably, from about 20A to about 70A. Preferably, the material of at least the distal end portion is flexible, e.g., elastic, stretchable or yieldable to allow the folds to partly or fully unfold.

According to another aspect of the present disclosure, the instrument introducer includes a body portion defining a lumen for passing a portion of the surgical instrument therethrough. The body portion includes a distal end portion fabricated from a flexible material and including a distal orifice, a proximal end portion including a proximal orifice, and at least one fold formed in at least the distal end portion of the body portion.

Desirably, the at least one fold provides the distal end portion with a taper extending in a distal direction. The distal end portion of the instrument introducer has a first condition having a generally conical shape. In the first condition, the distal end portion of the instrument introducer has a generally frusto-conical shape. Additionally, the distal end portion of the instrument introducer may be comprised of a plurality of folds extending radially about the distal end portion. Desirably, the folds extend in a longitudinal direction. In one embodiment, the folds provide the distal end portion with a taper extending in a distal direction.

The distal end portion of the instrument introducer has a first condition wherein the folds are unexpanded to facilitate insertion of the distal end portion into a body orifice. The distal end portion of the instrument introducer has one or more subsequent conditions wherein the folds are at least partially extended to accommodate the passage of a surgical instrument therethrough. The distal end portion of the instrument introducer has a subsequent condition having an extended internal diameter substantially equal to an outer diameter of a surgical instrument.

Desirably, the folds are integral with one another. It is envisioned that the folds may overlap one another. In one embodiment, the body portion of the instrument introducer has an inner diameter of from about 1.2 inches to about 1.6 inches.

The instrument introducer may be fabricated from at least one of a polypropylene, a moldable plastic, a thermoformable plastic, a polymer, a urethane, a silicone, a natural rubber, a synthetic rubber, an elastomer, an elastomeric material, and a latex material.

It is envisioned that the instrument introducer includes at least one fold which extends along the entire length of the body portion. The folds may be in the form of pleats. The body portion of the instrument introducer may include at least one annular fold formed about the body portion.

According to another aspect of the present disclosure, a method for inserting a distal end portion of a surgical instrument into a body opening of a patient, is provided. The method includes the step of providing an instrument introducer including a body portion defining a lumen therethrough for accommodating a distal end portion of a surgical instrument therethrough. The body portion may include a distal end portion having a distal orifice, a proximal end portion defining a proximal orifice, and at least one fold formed in at least the distal end portion of the body portion, wherein at least the distal end portion of the instrument introducer is fabricated from a flexible material.

The method further includes the steps of positioning the distal end portion of the instrument introducer into the body opening of a patient, inserting the distal end portion of the surgical instrument into the instrument introducer, and advancing the distal end portion of the surgical instrument through the lumen of the instrument introducer, such that as the distal end portion of the surgical instrument is passed into the distal end portion of the instrument introducer, the at least one fold radially expands to accommodate the distal end portion of the surgical instrument.

It is envisioned that the instrument introducer may include a plurality of folds extending radially around the distal end portion, and the plurality of folds may expand to accommodate the distal end portion of the surgical instrument. The folds may extend in a longitudinal direction such that during the advancing stage the folds gradually expand as the surgical instrument is passed into the distal end portion of the instrument introducer.

The distal end portion of the instrument introducer may have an initial condition wherein the folds are unexpanded and during the advancing step, the folds expand to facilitate insertion of the distal end portion of the instrument introducer into the body opening. Desirably, during the advancing step, the distal end portion of the instrument introducer may have a plurality of subsequent conditions wherein the folds are at least partially extended to accommodate movement of the distal end portion of the surgical instrument therethrough. Additionally, during the advancing step, the distal end portion of the instrument introducer may have an extended condition having an internal diameter substantially equal to an outer diameter of the distal end portion of the surgical instrument passing therethrough.

The method further includes the step of advancing the distal end portion of the surgical instrument through the distal orifice of the instrument introducer.

Desirably, at least the distal end portion of the instrument introducer is fabricated from at least one flexible material, and during the advancing step, the flexible material of the folds extend.

The method may further include the step of providing a lubricant on at least one of an inner surface of the instrument introducer and an outer surface of the distal end portion of the surgical instrument.

The presently disclosed instrument introducer, together with attendant advantages, will be more clearly disclosed below in the drawings and in the description of the drawings and of the preferred embodiments.

### BRIEF DESCRIPTION OF THE DRAWINGS

By way of example only, embodiments of the disclosure will be described with reference to the accompanying drawings, in which:

FIG. 1 is a top perspective view of an instrument introducer in accordance with an embodiment of the present disclosure;

FIG. 2 is a top plan view of the instrument introducer of FIG. 1;

FIG. 3 is a bottom plan view of the instrument introducer of FIGS. 1 and 2;

FIG. 4 is a side elevational view of the instrument introducer of FIGS. 1-3;

FIG. 5 is a cross-sectional side elevational view of the instrument introducer of FIGS. 1-4, as taken through a longitudinal axis thereof;

FIG. 6 is a perspective view of an instrument introducer in accordance with another embodiment of the present disclosure;

FIG. 7 is a side elevational view of the instrument introducer of FIG. 6;

FIG. 8 is a top plan view of the instrument introducer of FIGS. 6 and 7 prior to radial expansion thereof;

FIG. 9 is a top plan view of the instrument introducer of FIGS. 6-8 shown in a radially expanded condition with a surgical instrument extending therethrough;

FIG. 10 is a perspective view of an instrument introducer in accordance with another embodiment of the present disclosure;

FIG. 11 is a side elevational view of the instrument introducer of FIG. 10;

FIG. 12 is a side elevational view of the instrument introducer of FIGS. 10 and 11 whose body is in an extended and/or stretched state;

FIG. 13 is a side elevational view of the instrument introducer of FIGS. 10-12 in a bent and/or deflected state;

FIG. 14 is a top plan view of an instrument introducer in accordance with yet another embodiment of the present disclosure;

FIG. 15 is a top plan view of an instrument introducer in accordance with still another embodiment of the present disclosure;

FIG. 16 is a cross-sectional side elevational view, taken along the longitudinal axis, of the instrument introducer of FIGS. 1-5, depicting the initial insertion of a surgical instrument into the instrument introducer; and

FIG. 17 is a cross-sectional side elevational view, taken along the longitudinal axis, of the instrument introducer of FIGS. 1-5, depicting the complete insertion of a surgical instrument through the instrument introducer.

### DETAILED DESCRIPTION OF EMBODIMENTS

While the device according to the present disclosure is especially suitable for introducing a distal end portion of a surgical instrument into a body opening of a patient (for example, the anal orifice, the external urethral opening, the mouth or a natural or formed opening in a body cavity of a patient), for performing certain surgical procedures, (e.g., hemorrhoidal, mucosal prolapse or end-to-end anastomotic applications), it is envisioned that the device according to the present disclosure can be used in connection with other surgical instruments for performing any number of other endoscopic or laparoscopic surgical procedures. For example, it is believed that the device disclosed herein may find use in other procedures in which substantially blunt ended surgical instruments are introduced into body openings or cavities of patients.

In the drawings and in the description which follows, the term “proximal”, as is traditional, will refer to the end of the surgical device or instrument of the present disclosure which is closest to the operator, while the term “distal” will refer to the end of the device or instrument which is furthest from the operator.

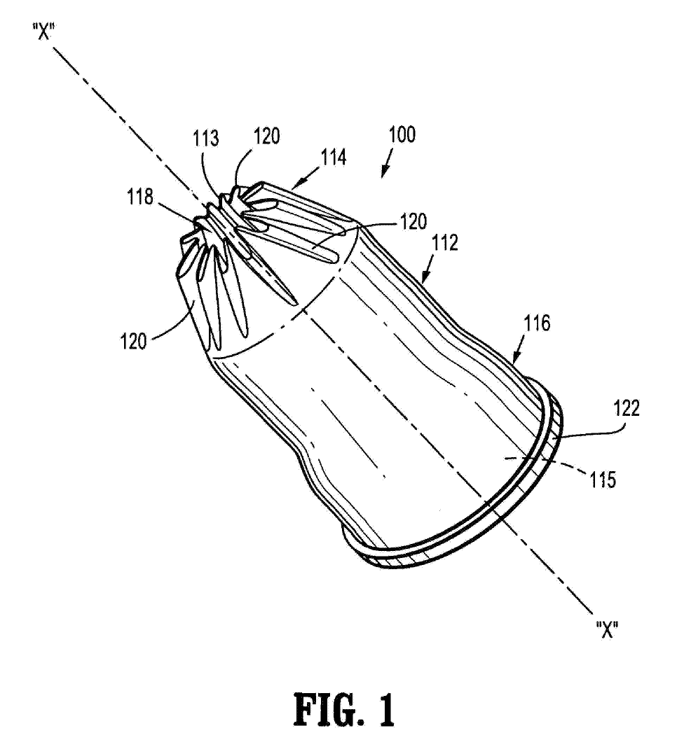

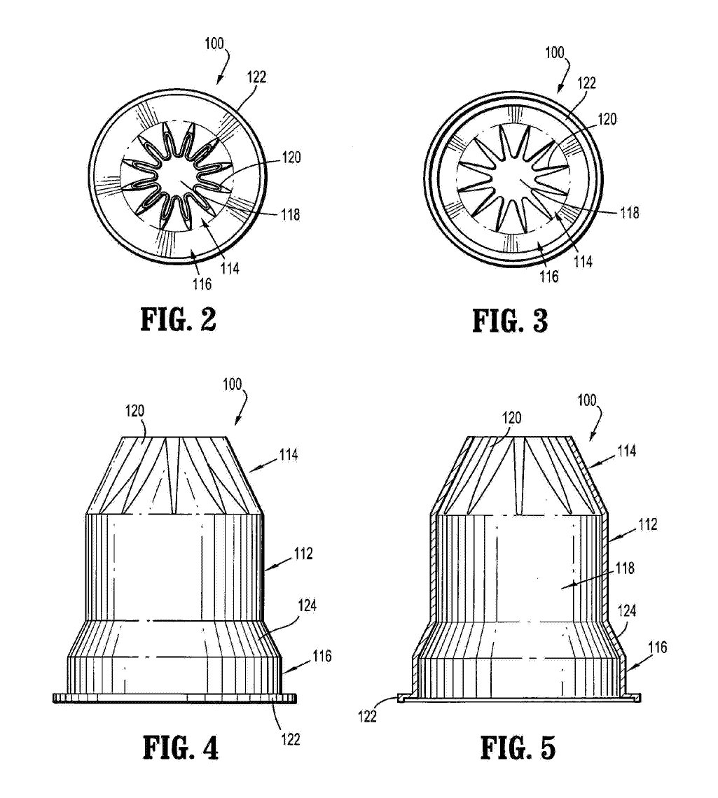

Referring now in specific detail to the drawings, in which like reference numerals identify similar or identical elements, FIGS. 1-4 illustrate a surgical instrument introducer generally designated as 100. Surgical instrument introducer 100 generally is in the form of a sheath, drape, sock, sleeve covering, casing, condom, etc. Accordingly, as used herein, instrument introducer 100 can embody any one of these terms.

As will be further described below, instrument introducer 100 is configured and adapted to be either removably mounted and/or placed on and/or over a distal end portion of a surgical instrument (e.g., anastomotic circular fastener applier or stapler) prior to insertion of the surgical instrument into an opening in the body, or is preferably configured and adapted for initial placement into the orifice of the body and for subsequent insertion of the distal end portion of the surgical instrument into and through the lumen of instrument introducer 100. Preferably, instrument introducer 100 has a shape which facilitates its entry into the orifice of the body and which is also compatible with the distal end portion of the surgical instrument. It is contemplated that instrument introducer 100 functions as a protective sheath or sleeve to cap, encase and/or surround the distal end portion of and to facilitate the entry of the distal end portion of the surgical instrument into the orifice of the body. In addition, instrument introducer 100 can act as an insulative barrier and/or an isolating barrier between the surgical instrument and the body of the patient.

As seen in FIGS. 1-5, instrument introducer 100 includes a hollow body portion 112 having a distal orifice 113, a distal end portion 114, a proximal orifice 115, and a proximal end portion 116. Body portion 112 defines a lumen 118 therethrough, which lumen 118 defines a central longitudinal axis “X”. Preferably, body portion 112 has an inner diameter sized to receive a distal end portion of a surgical instrument therein. For example, for a circular stapler, body portion 112 can have an inner diameter of about 1.2 to about 1.6 inches, preferably about 1.4 inches.

It is envisioned that body portion 112 can vary in length depending on the type of surgical instrument that is to be inserted into the orifice of the body through lumen 118 and depending on the particular surgical application in which instrument introducer 100 is going to be used. Preferably, for example, for a circular stapler, instrument introducer 100 will vary in overall length from about 2.0 inches to about 2.4 inches, preferably about 2.2 inches.

Distal end portion 114 of instrument introducer 100 includes at least one fold, preferably a plurality of folds 120 formed or extending radially thereabout such that distal end portion 114 tapers linearly or arcuately or a combination thereof, in a distal direction to a radially smaller diameter at or adjacent distal orifice 113. Preferably, the at least one fold or a plurality of folds extend in a longitudinal direction. The fold(s) can extend along the entire length of body portion 112. As used herein, the term “fold” is understood to include pleat, undulation, corrugation, crease, bend and the like. Generally, the fold(s) will be at or near distal orifice 113 such that the material that is folded, bent or undulating, etc., can open-up, unfold, extend or expand to allow the distal end of the instrument to pass through the orifice. Preferably, distal end portion 114 includes a plurality, e.g., an annular array, of folds 120 which in a first, e.g., at rest condition define a generally conical shape, more preferably, as shown, a generally or substantially frusto-conical configuration. While twelve folds 120 are shown, such should not be considered as limiting, as it is envisioned and within the scope of the present disclosure to have a distal end portion 114 including any number of folds greater or less than twelve. As shown in the Figures, the distal ends of the folds preferably are radially longer than their proximal ends. As will be described in greater detail below, the tapered configuration of distal end portion 114 facilitates insertion of instrument introducer 100 into the orifice of the patient.

Preferably, folds 120 are joined to or integral with one another and thus define a monolithic distal end portion 114. Alternatively, it is envisioned that folds 120 can be molded or formed to allow portions of folds 120 to extend over, overlie, overlap or rest on one another.

Distal end portion 114 has a first or initial configuration or condition, e.g., as formed or at rest, when no surgical instrument is extending therethrough, in which the fold or folds is or are unexpanded to facilitate insertion of the distal end portion into a body opening. In the initial condition, the portion of lumen 118 that extend through distal end portion 114 has a diameter which is less than the diameter of lumen 118 extending through body portion 112. Distal end portion 114 can have one or more subsequent or second configurations to accommodate the passage or extension of a surgical instrument therethrough, in which distal end portion 114 and/or distal orifice 113 is at least partially or fully extended or expanded to have a diameter which is substantially equal to the diameter of lumen extending through body portion 112. More particularly, in such configurations, distal orifice 113 or the portion of lumen 118 extending through body portion 112 has a diameter which is substantially equal to the outer diameter of the surgical instrument that is extending therethrough.

As seen in FIGS. 4 and 5, proximal end portion 116 preferably has a diameter which is larger than the radius of body portion 112 thereby defining a goose-neck portion 124 interconnecting body portion 112 with proximal portion 116. Proximal end portion has a size that facilitates insertion and entry of the surgical instrument into and through instrument introducer 100. For example, proximal end portion 116 can have an inner diameter of about 5.4 inches to about 9.4 inches, preferably about 7.4 inches.

The proximal end portion 116 of instrument introducer 100 preferably includes one or more radially outwardly extending portions such as one or more tabs or, more preferably, a radially outwardly extending continuous flange 122 integrally or monolithically formed with and/or connected to proximal end portion 116.

Flange 122 can be of any suitable length or width. For example, it can have one or more outwardly extending tabs, to provide one or more surfaces suitable for the placement of adhesives or tapes, e.g., such as those sold under the trademark Steri-Strip, or of sutures to fix or secure instrument introducer 100 and potentially the surgical instrument relative to the patient and to limit the depth of penetration of instrument introducer 100 and/or the distal end of the surgical instrument into the body of the patient. While it is envisioned that flange 122 is to be used when instrument introducer 100 is employed as a stand alone device that is unconnected to a surgical instrument, it is envisioned that an instrument introducer 100 having a flange 122 can also be employed when instrument introducer 100 is connected to the surgical instrument prior to insertion of instrument introducer 100 into the cavity or body opening of the patient.

Instrument introducer 100 can be of any suitable wall thickness. For example, it can have a wall thickness of about 0.03 inches to about 0.15 inches. Preferably, instrument introducer 100 has a uniform wall thickness throughout, however, the wall thickness can vary in portions or throughout.

Instrument introducer 100 can be fabricated from any suitable material or combinations of materials, for example, one or more moldable and/or thermoformable plastics, polymers, urethanes, natural or synthetic rubbers, silicones, elastomer and/or elastomeric or latex materials which is or are sufficiently extendible, expandible, pliable, malleable, ductile, compressible, elastic and/or rubbery to provide for controlled deflection and sufficient stiffness. Instrument introducer 100 is to be fabricated from an acceptable sterilizable medical grade material or combination of materials.

Instrument introducer 100 is preferably fabricated from a material having a reading on a Shore “A” durometer of generally about 20A to about 70A, but it could have a reading as low as 5A (the Shore “A” durometer reading for a hydrophillic contact lens) or about 90A (the Shore “A” durometer reading for a flexible acrylic material). Instrument introducer 100 can also be fabricated from a material having a reading on a Shore “D” durometer of about 40D to about 80D.

Other hardness scales can also be used. In general, durometers and their methods of use are described in ASTM D-2240. The Shore “A” scale can be used, for example, to measure the flexibility or relative hardness of synthetic rubbers, neoprene, silicones, felt, and the like. The Shore “B” scale can be used to measure the flexibility or alternatively the relative hardness of a variety of rubbers and elastomers. The Shore “C” scale is used to measure medium hard rubbers and plastics. The Shore “D” scale is used to measure a variety of plastics, Plexiglas, polystyrene, vinyls, and the like. The Shore “OO” scale can be used to measure the hardness of materials such as sponges, rubber or soft rubber. Durometers are available from Pacific Transducer Corp., Los Angeles, Calif.

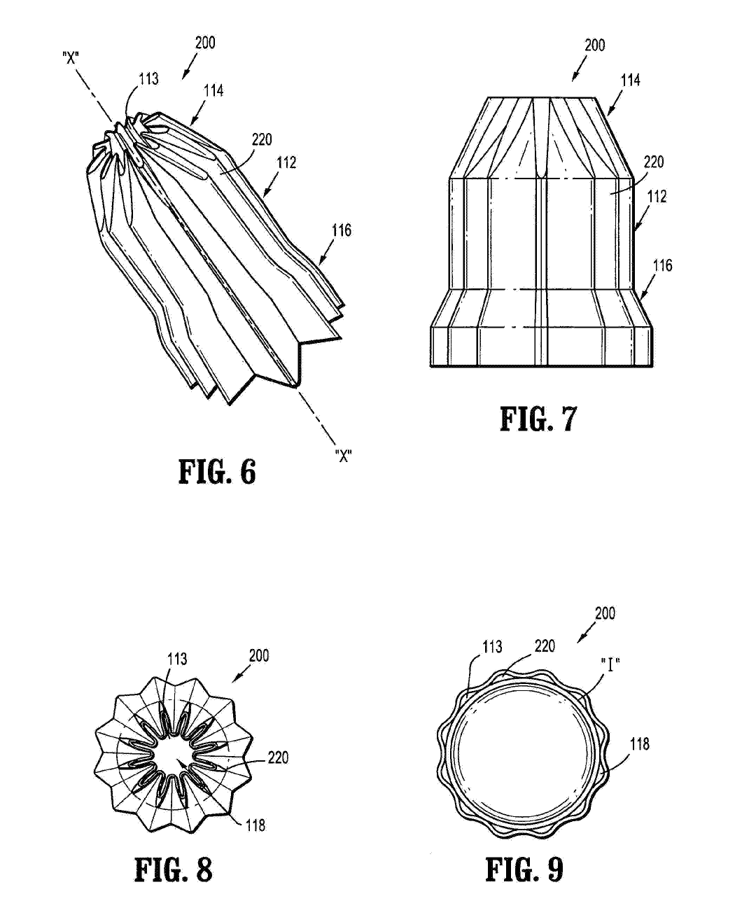

Turning now to FIGS. 6-9, an instrument introducer, in accordance with an alternative embodiment of the present disclosure, is generally designated 200. Instrument introducer 200 is similar to instrument introducer 100 and will only be described in detail to the extent necessary to identify differences in configuration, construction and/or operation.

As seen in FIGS. 6 and 7, instrument introducer 200 includes at least one, preferably a series of longitudinally oriented fold(s) 220, the series extending radially therearound. In an embodiment, folds 220 extend the entire length of instrument introducer 200 (i.e., from distal orifice 113, along distal end portion 114 and body portion 112, to proximal end portion 116). As such, the overall outer diameter of instrument introducer 200, and in this embodiment particularly of body portion 112, can be varied. In this manner, instrument introducer 200 can be inserted into orifices having relatively smaller diameters. In addition, as seen in FIG. 9, as a surgical instrument “I” is passed through distal orifice 113 and lumen 118 of instrument introducer 200, surgical instrument “I”, if having a diameter which is larger than the diameter of orifice 113 and/or the diameter of lumen 118 of instrument introducer 200, causes lumen 118 and in turn orifice 113, to radially extend or expand.

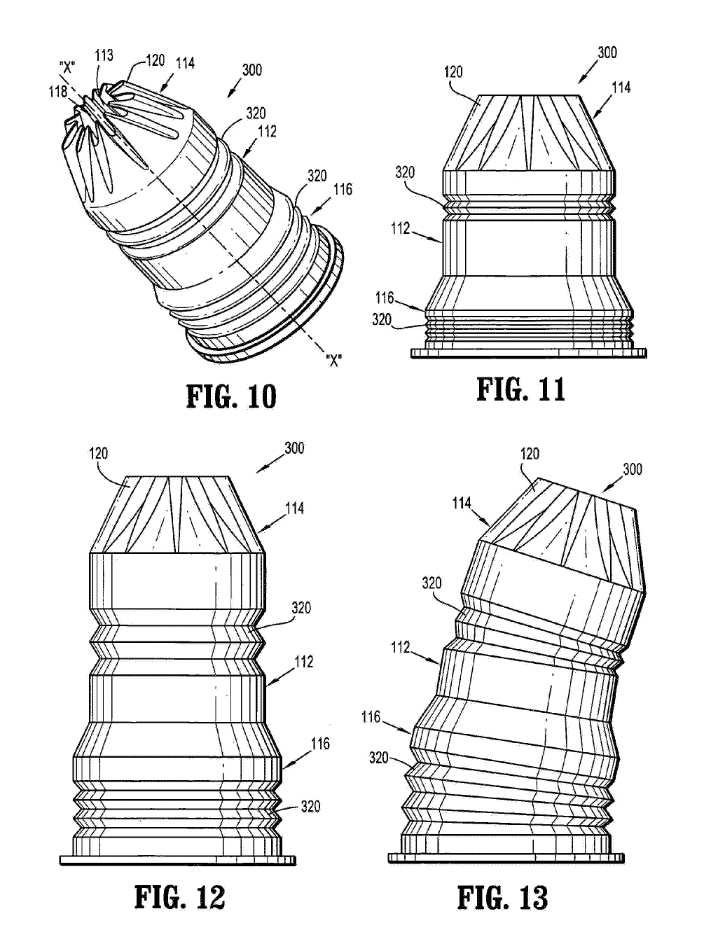

FIGS. 10-13 show another embodiment of an instrument introducer of the present disclosure, generally designated 300. Instrument introducer 300 is similar to instrument introducer 100 and will only be described in detail to the extent necessary to identify differences in configuration, construction and/or operation.

As seen in FIGS. 10-13, instrument introducer 300 includes at least one, preferably a plurality of, annular undulations or fold(s) 320, in the manner of an accordion, formed in body portion 112 and/or proximal end portion 116. In this manner, as seen in FIG. 12, as a surgical instrument is advanced through lumen 118 of instrument introducer 300, the friction between the inner surface of instrument introducer 300 and the outer surface of the surgical instrument, in addition to the forces required to advance the surgical instrument through and to radially expand distal end portion 114, causes instrument introducer 300 to extend and/or stretch distally, in a direction further into the body orifice. In addition, as seen in FIG. 13, annular fold(s) 320, formed in body portion 112 and/or proximal end portion 116, increase the overall flexibility of instrument introducer 300. In particular, folds 320 enable instrument introducer 300 to better conform (e.g., bend) to the shape or path of the body orifice and/or to better conform to the shape of the surgical instrument extending through lumen 118 thereof.

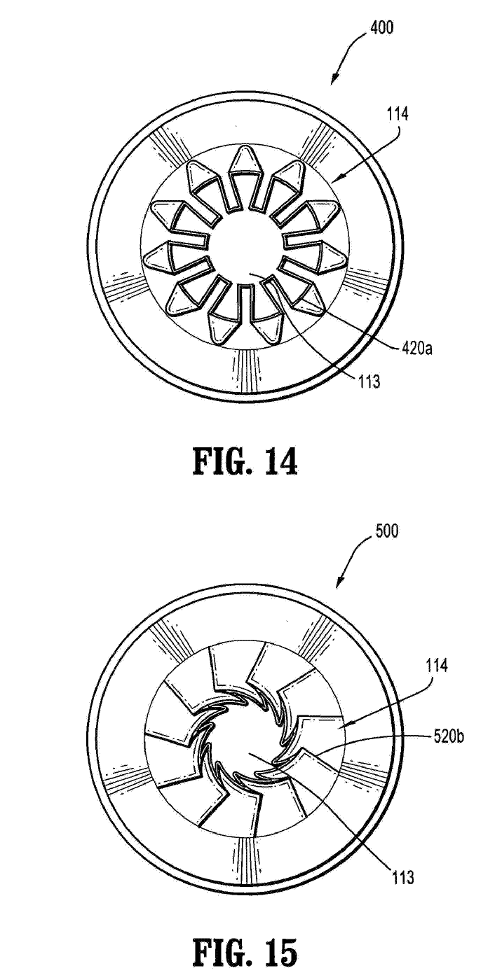

FIG. 14 shows another embodiment of an instrument introducer, generally designated 400. Instrument introducer 400 is similar to instrument introducer 100 and will only be described in detail to the extent necessary to identify differences in configuration, construction and/or operation.

As seen in FIG. 14, distal end portion 114 of instrument introducer 400 includes a plurality of pleats 420a, each having a substantially trapezoidal and/or triangular transverse cross-sectional profile, formed thereabout such that distal end portion 114 tapers in a distal direction to a radially smaller diameter.

FIG. 15 shows another embodiment of an instrument introducer, generally designated 500, having a distal end portion 114 that includes a plurality of pleats 520b, each having a folded-over and/or over-lapping configuration, formed therearound such that distal end portion 114 tapers in a distal direction to a radially smaller diameter.

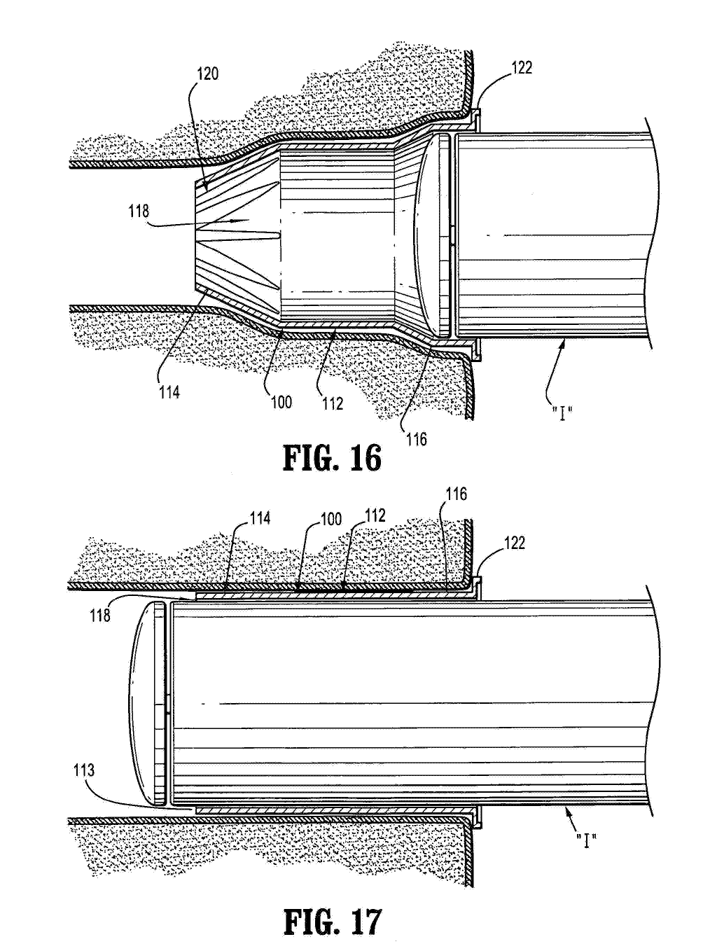

Use of instrument introducers 100-500 will now be described with reference to FIGS. 16 and 17. It is to be understood that use of each of instrument introducers 100-500 is substantially identical to one another and therefore, for the sake of clarity, simplicity and brevity, the following description will be directed to the use of instrument introducer 100.

In an embodiment of the method of use of an instrument introducer disclosed herein, instrument introducer, e.g., 100 is initially placed or positioned within a cavity or body opening or orifice of a patient (e.g., the anus) such that distal end portion 114 of instrument introducer 100 is retained therein at the desired depth. With at least distal end portion 114 of instrument introducer 100 in position, the distal end of a surgical instrument “I” is inserted into lumen 118 of instrument introducer 100 and advanced until the distal end of surgical instrument “I” contacts the inner surface of folds 120. As surgical instrument “I” is further advanced distally through instrument introducer 100, as seen in FIG. 17, the distal end portion of surgical instrument “I” begins to radially expand and/or stretch distal end portion 114 of instrument introducer 100, by unfolding one or more of folds 120, and to thereby radially enlarge lumen 118 of distal end portion 114 an amount sufficient to accommodate the passage of the distal end of surgical instrument “I” therethrough.

In an alternative method of use, instrument introducer 100 is initially placed on the distal end portion of surgical instrument “I”. With instrument introducer 100 positioned thereon, the distal end portion of surgical instrument “I” is inserted and/or introduced into the cavity, orifice and/or body opening of the patient to the desired depth and/or position. With instrument introducer 100 held at the desired depth, surgical instrument “I” is distally advanced relative to instrument introducer 100 causing distal end portion 114 to radially expand and/or stretch. Distal end portion 114 radially expands an amount sufficient to accommodate the passage of the distal end of surgical instrument “I” therethrough.

In either method, it is understood by those skilled in the art that flange 122 will limit the depth of penetration of instrument introducer 100 into the body orifice of the patient. Additionally, in either method, it is preferred that a lubricant (e.g., petroleum jelly, water-based lubricants, glycerin, etc.), compatible with the materials of the introducer, surgical instrument and patient, is applied to the inner surface of instrument introducer 100 and/or the outer surface of surgical instrument “I” in order to facilitate insertion of surgical instrument “I” through instrument introducer 100. Additionally, it is contemplated that a suitable lubricant can be applied to the outer surface of instrument introducer 100 and/or into the cavity or body orifice of the patient in order to facilitate insertion of instrument introducer 100 and/or surgical instrument “I” into and through instrument introducer 100.

Although the illustrative embodiments of the present disclosure have been described herein, it is understood that the disclosure is not limited to those precise embodiments, and that various other changes and modifications may be affected therein by one skilled in the art without departing from the scope or spirit of the disclosure. All such changes and modifications are intended to be included within the scope of the disclosure.

1. (canceled)

2. An instrument introducer comprising:
a body portion defining a lumen therethrough and having an inner diameter sized to receive a distal end portion of a surgical instrument therein, the lumen defining a central longitudinal axis; and
a folded portion supported on a distal end of the body portion, the folded portion including a plurality of folds in an overlapping annular configuration, the plurality of folds extending radially inwardly of the body portion to a radial inner edge defining an opening therethrough.

3. The instrument introducer according to claim 2, wherein the opening of the folded portion is coincident with the central longitudinal axis.

4. The instrument introducer according to claim 2, wherein the folded portion has an unexpanded condition in which the opening of the folded portion has an unexpanded diameter smaller than the inner diameter of the body portion.

5. The instrument introducer according to claim 4, wherein the folded portion has an expanded condition in which the opening of the folded portion has an extended diameter substantially equal to an outer diameter of a surgical instrument.

6. The instrument introducer according to claim 2, wherein the folded portion is a monolithic structure.

7. The instrument introducer according to claim 6, wherein the folded portion includes rubber.

8. The instrument introducer according to claim 6, wherein the folded portion includes silicone.

9. The instrument introducer according to claim 2, wherein portions of the plurality of folds of the folded portion abut one another.

10. The instrument introducer according to claim 2, wherein each fold of the plurality of folds includes an outer fold region and an inner fold region, the outer fold region transitioning to the inner fold region at an outer folded edge.

11. The instrument introducer according to claim 10, wherein the inner fold region is positioned under a portion of the outer fold region.

12. The instrument introducer according to claim 10, wherein the plurality of folds are interconnected at inner folded edges, each inner folded edge formed between the outer fold region of one fold of the plurality of folds and an inner fold region of an adjacent fold.

13. The instrument introducer according to claim 12, wherein the inner fold region of each fold of the plurality of folds overlaps a portion of the outer fold region of an adjacent fold.

14. The instrument introducer according to claim 10, wherein the outer folded edges of the plurality of folds are oriented in the same direction.

15. The instrument introducer according to claim 2, wherein the folded portion extends distally form a distal end portion of the body portion.
